(19) United States
(12) Patent Application Publication
(10) Pub. No.: US 2007/0065471 A1
(43) Pub. Date: Mar. 22, 2007

(54) Cosmetic/dermatological applications of PPAR receptor activators

(75) Inventors: Andre Jomard, Saint Vallier De Thiey (FR); Michel Rivier, Nice (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT S.N.C., VALBONNE SOPHIA ANTIPOLIS (FR)

(21) Appl. No.: 11/444,371

(22) Filed: Jun. 1, 2006

Related U.S. Application Data
(63) Continuation of application No. PCT/FR04/03069, filed on Nov. 30, 2004.

(30) Foreign Application Priority Data
Dec. 1, 2003 (FR) 03/14082

(51) Int. Cl. A61K 8/49; A61K 8/46; A61K 31/427; A61K 31/195

(52) U.S. Cl. 424/401; 514/342; 514/369; 514/563; 514/562

(57) ABSTRACT

Cosmetic/pharmaceutical compositions useful for regulating the size of sebaceous glands and, in particular, for inhibiting sebum production, or for treating pathologies entailing an overproduction of sebum, or for treating greasy skin and/or a scalp prone to dandruff, contain thus effective amounts of at least one activator of PPAR type receptors.

### CROSS-REFERENCE TO PRIORITY/PCT APPLICATIONS

This application claims priority under 35 U.S.C. § 119 of FR 03/14082, filed Dec. 1, 2003, and is a continuation of PCT/FR 2004/003069, filed Nov. 30, 2004 and designating the United States (published in the French language on Jun. 16, 2005 as WO 2005/053632 A2; the title and abstract were also published in English), both hereby expressly incorporated by reference and both assigned to the assignee hereof.

### BACKGROUND OF THE INVENTION

1. Technical Field of the Invention

The present invention relates to the formulation of at least one activator of PPAR type receptors into a cosmetic composition or for the preparation of a pharmaceutical composition, the said PPAR receptor activator or the said composition being useful to regulate the size of the sebaceous glands.

In particular, the said PPAR receptor activator or the said composition is useful to inhibit the production of sebum by the sebaceous glands.

The present invention also relates to the use of the said PPAR receptor activator for the preparation of pharmaceutical compositions for treating pathologies associated with an overproduction of sebum, and also to the formulation of the said activator into cosmetic compositions, as a matting agent or as an anti-dandruff agent.

2. Description of Background and/or Related and/or Prior Art

Sebum is a holocrine secretion from the cells of the sebaceous gland, or sebocytes. Maturation of the sebocytes is characterized by the production of lipids. Human sebum consists predominantly of triglycerides, waxes, squalene, cholesterol esters, fatty acids and other lipids in smaller amounts. Many factors may affect the secretion of sebum.

The sebaceous glands are generally associated with the follicles of head hair and other bodily hairs and also with their function, thus forming a pilosebaceous unit. They exist throughout the body and are more particularly concentrated on the face, the forehead and the scalp. Certain sebaceous glands are not associated with head hair and other bodily hairs. All sebaceous glands, irrespective of the animal species from which they are derived, have a similar structure. They consist of a single lobule or acinus, or of a collection of lobules that open onto a hair duct. The sebaceous glands alone themselves open directly onto the surface of the skin.

Sebocytes are specialized epithelial cells that proliferate first in an undifferentiated state and then become differentiated in the basal and parabasal layers (Mednieks et al., J. Invest. Dermatol., 97: 517-523, 1991). The differentiation takes place as lipid-charged cells which, at the end of maturation, finish by breaking and releasing the sebum by holocrine secretion.

Disorders associated with sebaceous function may result in aesthetic disorders: this is the case for greasy skin, which is characterized by exaggerated secretion and excretion of sebum. Such skin has a shiny, thick appearance, the follicular orifices of which are dilated or filled with minute horny spicules, or even with comedones. Greasy skin is often associated with a desquamation defect, and with a thick skin grain. The excess sebum may also serve as a support for the anarchic growth of the bacterial flora and cause comedones and/or acne lesions, or alternatively on the scalp it may cause abnormal desquamation associated with the presence of the yeast Malassezia, which is responsible for the production of dandruff.

Disorders associated with sebaceous function may also result in dermatological disorders, especially perioral dermatitis, pathologies associated with hyperplasia of the sebaceous glands such as hereditary hyperplasia of the sebaceous glands, and the overproduction of sebum associated with hormonal disorders such as hyperandrogeny of endocrine origin.

The prior art describes the use of agents for absorbing sebum, or of keratolytic agents such as α- or β-hydroxy acids, active agents which act directly on the production of sebum.

Need nevertheless exists for compounds that are effective for avoiding an overproduction of sebum and its aesthetic and dermatological consequences.

### SUMMARY OF THE INVENTION

It has now surprisingly been found that the administration of a PPAR (Peroxisome Proliferator Activated Receptor) activator has the consequence of reducing the size of the sebaceous glands and thus of reducing the overall production of sebum, contrary to the teachings of the prior art.

WO 98/08089 by Arch Development demonstrates, specifically, that PPARgamma agonists such as thiazolidinediones stimulate sebum production in a culture of preputial sebocytes, and thus proposes, in contrast with the present invention, the administration of antagonists of these receptors to inhibit the production of sebum by the sebaceous glands.

Peroxisomes are small organites similar to mitochondria, containing a series of enzymes specific for the metabolism of hydrogen peroxide (catalase, urate oxidase, D-amino acid oxidase) and enzymes for the β-oxidation of fatty acids. Peroxisome proliferators are mainly groups of chemical products that comprise hypolipidaemiant agents, such as clofibrate, herbicides and industrial plastics, such as phthalate esters. These peroxisome proliferators activate receptors, known as the PPARs, which form part of the superfamily of steroidal nuclear receptors. These receptors may be activated by the peroxisome proliferators, and may also be activated by natural fatty acids, and thus stimulate the expression of genes coding for the enzymes involved in peroxisomal and mitochondrial β-oxidation or for P450-4A6 α-hydroxylase of fatty acid.

The PPAR receptors activate transcription by binding to DNA sequence elements, known as peroxisome proliferator response elements (PPREs), in the form of a heterodimer with the retinoid X receptors (known as the RXRs).

Three subtypes of human PPAR receptor have been identified and described: PPARalpha (α), PPARgamma (γ) and PPARdelta (δ) (or NUC1).

Thus, the present invention features the formulation of at least one activator of PPAR type receptors into cosmetic compositions or into pharmaceutical compositions, the said PPAR receptor activator or said compositions being useful to regulate the size of the sebaceous glands.

In particular, the said PPAR receptor activator or the said composition is useful to inhibit the production of sebum.

The cosmetic or pharmaceutical compositions according to the invention comprise a physiologically acceptable medium.

The term “physiologically acceptable medium” means a medium that is compatible with the skin and optionally with its integuments (eyelashes, nails, hair) and/or mucous membranes.

According to the invention, the term “PPAR receptors” especially means the subtypes PPAR-α, PPAR-δ and PPAR-γ.

The compounds according to the invention exhibit activating properties on PPAR type receptors. This activating activity on the PPAR receptors may be measured in a transactivation test by means of the dissociation constant Kdapp (apparent).

According to the invention, the term “activator of PPAR type receptors” especially means any agonist compound that binds to the PPAR receptor and that has, for at least one of the subtypes PPAR α, γ, or δ, a dissociation constant Kdapp of less than or equal to 1 μM, in a transactivation test as described in Example 1 to follow.

### DETAILED DESCRIPTION OF BEST MODE AND SPECIFIC/PREFERRED EMBODIMENTS OF THE INVENTION

The preferred compounds of the present invention have, for at least one of the subtypes PPAR α, γ or δ, a dissociation constant Kdapp of less than or equal to 500 nM and advantageously less than or equal to 100 nM.

A PPAR activator having, for at least the subtype PPAR-γ, a dissociation constant Kdapp of less than or equal to 500 nN and advantageously less than or equal to 100 nM is even more preferred.

Examples of such a compound that are illustrative include the following:
1. 5-{4-2-methylpyrid-2-ylamino)ethoxy]benzyl}-thiazolidine-2,4-dione, which is of subtype α and γ,
8. 2-(4-{2-[3-(2,4-difluorophenyl)-1 -heptylureido]-ethyl}phenylsulfanyl)-2-methylpropionic acid.

Even more preferably, the activator of PPAR-γ type receptors is specific, i.e., it has a ratio R of Kdapp relative to PPAR-γ to the Kdapp relative to PPARα of less than or equal to 10⁻¹. Preferably, R is less than or equal to 0.05 and more advantageously less than or equal to 0.02.

Examples of specific activators of PPAR-γ receptors that are illustrative include the following:
2. N-methyl-N-[4′-(2,4-dioxothiazolidin-5-ylmethyl)-biphenyl-3-ylmethyl)-6-(2-methoxyethoxymethoxy)-naphthalene-2-carboxamide,
3. (S)-3-[3′-(3-heptyl-1 -methylureido)biphenyl-4-yl]-2-[2-(1-phenylmethanoyl)phenylamino]propionic acid,
4. N-methyl-N-[4′-(2,4-dioxothiazolidin-5-ylmethyl)-biphenyl-3-ylmethyl]-6-propoxynaphthalene-2-carboxamide,
5. (S)-2-ethoxy-3-{3′-[(methyloctanoylamino)methyl]biphenyl-4-yl}propionic acid,
6. 2-{3′-[({1-[6-(2-methoxyethoxymethoxy)naphthalene-2-yl]methanoyl}methylamino)methyl]biphenyl-4-ylamino}-benzoic acid,
7. (S)-3-{3′-[3-(4-dimethylaminophenyl)-1-methyl-ureido]biphenyl-4-yl}-2-[2-(1-phenylmethanoyl)phenyl-amino]propionic acid,
9. 1-[4′-(2,4-dioxothiazolidin-5-ylmethyl)biphenyl-3-yl]-3-heptyl-1-methylurea,
10. {3-[4-(3-cyclohexyl-1-phenethylureido)phenyl-sulfanyl]phenyl}acetic acid,
11. {3-[4-(3-hexyl-1-phenethylureido)phenylsulfanyl]-phenyl}acetic acid,
12. {3-[4-(1-butyl-3-cyclohexylureido)phenylsulfanyl]-phenyl)acetic acid,
13. {3-[4-(3-benzyl-1-butylureido)phenylsulfanyl]-phenyl}acetic acid,
14. (S)-3-[3′-(3-heptyl-1-methylureido)biphenyl-4-yl]-2-[2-(1-phenylmethanoyl)phenylamino]propionic acid,
15. Ethyl (S)-3-[3′-(3-heptyl-1-methylureido)biphenyl-4-yl]-2-[2-(1-phenylmethanoyl)phenylamino]propionate.

Other characteristics, aspects, subjects and advantages of the invention will emerge even more clearly from the description that follows, and also the various concrete, but in no way limiting, illustrative examples.

The administration (regime or regimen) of the compositions according to the invention may be performed orally, parenterally or topically. The composition is preferably packaged in a form suitable for topical or oral application, preferentially topical application.

Via the oral route, the composition, more particularly the pharmaceutical composition, may be in the form of tablets, gel capsules, sugar-coated tablets, syrups, suspensions, solutions, powders, granules, emulsions, or lipid or polymer microspheres, nanospheres or vesicles allowing a controlled release. Via the parenteral route, the composition may be in the form of solutions or suspensions for infusion or for injection.

The compounds according to the invention are generally administered at a daily dose of about from 0.001 mg/kg to 100 mg/kg of body weight in 1 to 3 dosage intakes.

Via the topical route, the compositions according to the invention are more particularly useful for treating the skin and mucous membranes, and may be in the form of ointments, creams, milks, pomades, powders, impregnated pads, syndets, solutions, gels, sprays, foams, suspensions, lotions, sticks, shampoos or washing base. They may also be in the form of suspensions of lipid or polymer microspheres, nanospheres or vesicles, or in the form of polymer patches and hydrogels allowing a controlled release. This topical composition may be in anhydrous form, in aqueous form or in the form of an emulsion.

The compounds are used topically at a concentration generally of from 0.001% to 10% by weight and preferably from 0.01% to 1% by weight relative to the total weight of the composition.

The cosmetic and dermatological compositions as described above may also contain inert or pharmacodynamically active additives, as regards pharmaceutical compositions, or combinations of these additives, and especially:
wetting agents;
flavor enhancers;
preservatives such as para-hydroxybenzoic acid esters;
stabilizers;
humidity regulators;
pH regulators;
osmotic pressure modifiers;
emulsifiers;
UV-A and UV-B screening agents;
desquamating agents;
anti-oxidants, such as α-tocopherol, butylhydroxyanisole or butylhydroxytoluene, superoxide dismutase, ubiquinol or certain metal-chelating agents;
depigmenting agents such as hydroquinone, azeleic acid, caffeic acid or kojic acid;
emollients;
moisturizers, for instance glycerol, PEG 400, thiamorpholinone and derivatives thereof, or urea;
anti-seborrhoeic or anti-acne agents, such as S-carboxymethylcysteine, S-benzylcysteamine, salts thereof or derivatives thereof, or benzoyl peroxide;
antibiotics, for instance erythromycin and itsesters, neomycin, clindamycin and its esters, and tetracyclines;
anti-fungal agents such as ketoconazole or 4,5-poly-methylene-3-isothaizolidones;
non-steroidal anti-inflammatory agents;
anti-psoriatic agents such as anthralin and its derivatives;
eicosa-5,8,11,14-tetraynoic acid and eicosa-5,8,11-triynoic acid, and esters and amides thereof;
retinoids, i.e., natural or synthetic ligands of the RAR or RXR receptors;
corticosteroids or oestrogens;

α-hydroxy acids and α-keto acids or derivatives thereof, such as lactic acid, malic acid, citric acid, glycolic acid, mandelic acid, tartaric acid, glyceric acid or ascorbic acid, and also salts, amides or esters thereof, or β-hydroxy acids or derivatives thereof, such as salicylic acid and the salts, amides or esters thereof;
ion-channel blockers such as potassium-channel blockers;
agents for combating desquamative conditions of the scalp, for instance zinc pyrithione, piroctone olamine, selenium disulfide, climbazole, undecylenic acid, ketoconazole, piroctone olamine (octopirox) or ciclo pirocton (ciclopirox), in particular for the “anti-dandruff compositions;
other matting agents, such as powders or agents consisting of colloidal dispersions of mineral particles, for instance silica, in particular for the “matting” cosmetic compositions;
or, alternatively, more particularly for pharmaceutical compositions, in combination with medicaments already known to interfere with the immune system (for example cyclosporine, FK 506, gluco corticoids monoclonal antibodies, cytokines or growth factors, etc.).

Needless to say, one skilled in this art will take care to select the optional compound(s) to be added to these cosmetic or dermatological compositions according to the desired effect (matting, anti-dandruff, or intended for treating pathologies associated with hyperproduction of sebum), and such that the advantageous properties intrinsically associated with the present invention are not, or are not substantially, adversely affected by the envisaged addition.

The present invention features the formulation of at least one activator of PPAR type receptors as defined above, for the preparation of a pharmaceutical composition for treating perioral dermatitis, pathologies associated with hyperplasia of the sebaceous glands, such as hereditary hyperplasia of the sebaceous glands, or pathologies of overproduction of sebum associated with hormonal disorder such as hyperandrogeny of endocrine origin.

This invention also features the cosmetic applications of at least one activator of PPAR type receptors as defined above, as a matting agent or alternatively as an anti-dandruff agent.

The present invention also features a cosmetic regime or regimen for treating greasy skin, wherein a composition comprising at least one activator of PPAR type receptors as defined above is administered or topically applied to the skin, mucous membranes or keratin fibers.

This invention also features a cosmetic regime or regimen for preventing and/or treating a scalp with a tendency towards dandruff, wherein a composition comprising at least one activator of PPAR type receptors as defined above is administered or topically applied to the skin, mucous membranes or keratin fibers.

The compositions according to the invention may be administered orally or applied locally to the areas to be treated. The administration or application may be performed daily, for a duration of several weeks, and the treatment may be renewed periodically, according to the individual to be treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Results of the PPAR Transactivation Test

Activation of the PPAR receptors with an agonist (activator) in HeLN cells leads to the expression of a reporter gene, luciferase, which, in the presence of a substrate, generates light. The modulation of the PPAR receptors is measured by quantifying the luminescence produced after incubating the cells in the presence of a reference agonist. The ligands displace the agonist from its site. The measurement of the activity is performed by quantifying the light produced. This measurement makes it possible to determine the modulatory activity of the compounds according to the invention by determining the constant that represents the affinity of the molecule for the PPAR receptor. This value can fluctuate according to the basal activity and the expression of the receptor: it is referred to as Kd apparent (KdApp in nM).

To determine this constant, the cells are in contact with a concentration of the test product and a concentration of the reference agonist, 2-(4-{2-[3-(2,4-difluorophenyl)-1-heptylureido)ethyl}phenyl-sulfanyl) -2-methylpropionic acid for PPARα, {2-methyl-4-[4-methyl-2-(4-trifluoromethylphenyl) thiazol-5-yl-methylsulfanyl]phenoxy}acetic acid for PPARδ and 5-{4-[2-methylpyrid-2-ylamino)ethoxy]benzyl}thiazolidine-2,4-dione for PPARγ. Measurements are also taken for the total agonist controls with the same products.

The HeLN cell lines used are stable transfectants comprising the plasmids ERE-βGlob-Luc-SV-Neo (reporter gene) and PPAR (α, δ, γ) Gal-hPPAR. These cells are inoculated in 96-well plates at a rate of 10,000 cells per well in 100 μ1 of DMEN medium without phenol red and supplemented with 10% defatted calf serum. The plates are then incubated at 37° C. and 7% CO₂ for 16 hours.

The various dilutions of the test products and of the reference ligand are added in a proportion of 5 μ1 per well. The plates are then incubated for 18 hours at 37° C., 7% CO₂. The culture medium is removed by upturning and 100 μl of a 1:1 PBS/luciferin mixture are added to each well. After 5 minutes, the plates are read using a luminescence reader.

The test compounds, referred to as Examples 1 to 8 in the following table, are:

### EXAMPLE 1

### 5-{4-[2-(methylpyrid-2-ylamino)ethoxy]-benzyl}thiazolidine-2,4-dione,

### EXAMPLE 2

### N-methyl-N-[4′-(2,4-dioxothiazolidin-5-yl-methyl)biphenyl-3-ylmethyl]-6-(2-methoxyethoxymethoxy)-naphthalene-2-carboxamide,

### EXAMPLE 3

### (S)-3-[3′-(3-heptyl-1-methylureido)biphenyl4-yl]-2-[2-(1-phenylmethanoyl)phenylamino]-propionic acid,

### EXAMPLE 4

### N-methyl-N-[4′-(2,4-dioxothiazolidin-5-yl-methyl)biphenyl-3-ylmethyl)-6-propoxynaphthalene-2-carboxamide,

### EXAMPLE 5

### (S)-2-ethoxy-3-{3′-[(methyloctanoylamino)methyl]biphenyl-4-yl}propionic acid,

### EXAMPLE 6

### 2-{3′-[({1-[6-(2-methoxyethoxymethoxy)-naphthalen-2-yl]methanoyl}methylamino)methyl]biphenyl-4-ylamino}benzoic acid,

### EXAMPLE 7

### (S)-3-{3′-[3(4-dimethylaminophenyl)-1-methylureido]biphenyl-4-yl}-2-[2-(1-phenylmethanoyl)-phenylamino]propionic acid,

### EXAMPLE 8

### 2-(4-{2-[3-(2,4-difluorophenyl)-1-heptylureido)ethyl}phenylsulfanyl)-2-methylpropionic acid.

Transactivation results:

| | PPAR alpha | PPARs delta | PPAR gamma |
|---|---|---|---|
| Compounds | Kd app (nM) | Kd app (nM) | Kd app (in nM) |
| Reference 1 PPARα: | 200 | n.a. | n.a. |
| Reference 2 PPARδ: | n.a. | 10 | n.a. |
| Reference 3 PPARγ: | n.a. | n.a. | 30 |
| Example 1 | 250 | 600 | 250 |
| Example 2 | n.a. | n.a. | 0.5 |
| Example 3 | n.a. | 250 | 15 |
| Example 4 | n.a. | n.a. | 1 |
| Example 5 | 500 | 4000 | 2 |
| Example 6 | n.a. | n.a. | 30 |
| Example 7 | n.a. | 4000 | 8 |
| Example 8 | 250 | n.a. | 250 |

n.a. means not active

These results show the affinity of the compounds for the PPAR receptors and more particularly the specificity of the affinity of certain compounds of the invention for the PPARγsubtype, and of others for the PPARα subtype.

### EXAMPLE 2

### Evaluation of the Activity of PPAR Activators on the Size of Sebaceous Glands

The evaluation of the activity of the compounds according to the invention is performed by daily topical application (once a day, weekends included) to the skin of the back of female Fuzzy or OFA rats, for 4 weeks. 30 animals of 9/10 weeks old were divided into groups of 6 animals.

The evaluation method entails weighing the animals at the start and end of the test and in evaluating the size of the sebaceous glands on epidermal leaflets.

The animals are euthanized and the treated area (back) is then shaved and defatted. The 8 mm biopsy samples are then incubated in 1 M NaBr. After separating out the epidermis, photographs are taken of the sebaceous glands and the images obtained are then analyzed using the Tina software (quantification of the area of the sebaceous glands, arbitrary area unit [mm²]), version 2.09 g sold by Raytest GmbH.

The statistical analysis is performed according to Student's t test (XL software sold by Microsoft)

Results:

4 studies were performed with the PPARγ agonist compounds numbered 1 to 3 in Example 1, according to the method described above in comparison with a positive control represented by the androgen 5a-androstan-17β-ol-3-one by the androgen (stimulator of sebum production) and a negative control represented by a PPARgamma antagonist compound, 1-{2-[(S)-2-{4-[2-(5-methyl-2-phenyloxazol-4-yl)ethoxy]phenyl}-1-(5-propyl-[1,3,4]oxadiazol-2-yl)ethylamino)phenyl}-1-phenyl-methanone.

In the control, the animals are not treated with any active principle: the control is acetone.

The results are expressed as the mean of the inductions (“Mean RI”) with the value of the standard error of the mean (“SEM”).

Student's t test (“stat”): NS not significant
*p<0.05
**p<0.01
***p<0.001

Positive Control (Stimulates Sebum Secretion):
5a-androstan-17β-ol-3-one

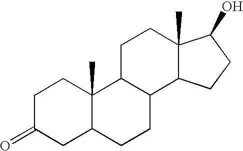

### EXAMPLE 1

### 5-{4-[2-(methylpyrid-2-ylamino)ethoxy]benzyl}thiazolidine-2,4-dione

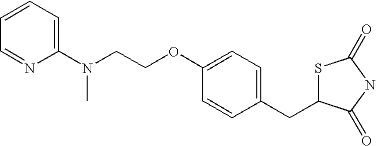

### EXAMPLE 2

### N-methyl-N-[4′-(2,4-dioxothiazolidin-5-yl -methyl)biphenyl-3-ylmethyl]-6-(2-methoxyethoxymethoxy) naphthalene-2-carboxamide

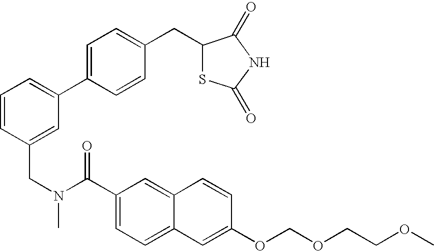

### EXAMPLE 3

### (S)-3-[3,-(3-heptyl-1-methylureido)biphenyl-4-yl]-2-[2-(1-phenylmethanoyl)phenylamino]propionic acid

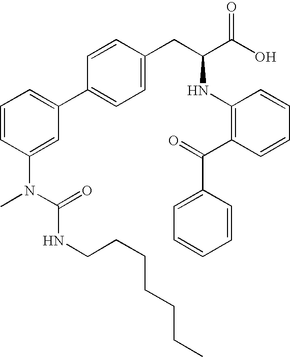

Negative control: (PPARγ antagonist): 1-{2-[(S)-2-{4-[2-(5-methyl-2-phenyloxazol-4-yl)ethoxy]phenyl}-1-(5-propyl[1,3,4]oxadiazol-2-yl)ethylamino]phenyl}-1-phenylmethanone

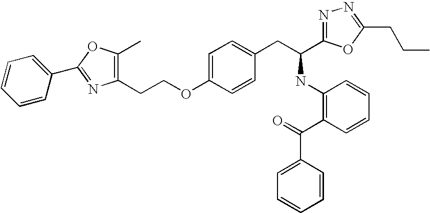

Study 1:

| Compound | Mean RI | SEM | stat |
|---|---|---|---|
| Control | 1.00 | 0.09 | — |
| Positive Control (0.3%) | 1.73 | 0.08 | *** |
| Example 1 0.1% | 0.73 | 0.06 | NS |
| Example 1 0.3% | 0.55 | 0.04 | * |
| Example 1 1% | 0.51 | 0.02 | ** |

Study 2:

| Compound | Mean RI | SEM | stat |
|---|---|---|---|
| Control | 1.00 | 0.05 | — |
| Example 1 0.1% | 0.83 | 0.03 | * |
| Example 2 0.003% | 0.82 | 0.05 | * |
| Example 2 0.03% | 0.75 | 0.03 | ** |
| Example 2 0.3% | 0.65 | 0.03 | *** |

Study 3:

| Compound | Mean RI | SEM | stat |
|---|---|---|---|
| Control | 1.00 | 0.03 | — |
| Example 2 0.3% | 0.66 | 0.06 | *** |
| Example 3 0.01% | 1.02 | 0.07 | NS |
| Example 3 0.1% | 0.84 | 0.07 | NS |
| Example 3 1% | 0.74 | 0.05 | *** |

Study 4:

| Compound | Mean RI | SEM | stat |
|---|---|---|---|
| Control | 1.00 | 0.03 | — |
| Example 2 0.3% | 0.67 | 0.06 | *** |
| Negative Control 0.01% | 1.12 | 0.05 | NS |
| Negative Control 0.1% | 1.08 | 0.09 | NS |
| Negative Control 1% | 1.08 | 0.03 | NS |

These results clearly show that the PPAR-activating compounds, in particular the specific PPARγ activators, significantly reduce the size of sebaceous glands compared with that obtained with the positive control, in contrast with the PPARgamma antagonist.

### EXAMPLE 3

### Specific Formulations

In this example, various specific formulations based on compounds according to the invention are illustrated.

A - ORAL ROUTE:

| | | |
|---|---|---|
| (a) 0.2 g Tablet: | | |
| Compound of Example 1 | 0.001 | g |
| Starch | 0.114 | g |
| Dicalcium phosphate | 0.020 | g |
| Silica | 0.020 | g |
| Lactose | 0.030 | g |
| Talc | 0.010 | g |
| Magnesium stearate | 0.005 | g |
| (b) Drinkable suspension in 5 ml ampules: | | |
| Compound of Example 2 | 0.001 | g |
| Glycerol | 0.500 | g |
| 70% sorbitol | 0.500 | g |
| Sodium saccharinate | 0.010 | g |
| Methyl para-hydroxybenzoate | 0.040 | g |
| Flavoring | qs | |
| Purified water | qs 5 | ml |

B TOPICAL ROUTE:

| | | |
|---|---|---|
| (a) Ointment: | | |
| Compound of Example 6 | 0.300 | g |
| White petroleum jelly codex | qs 100 | g |
| (b) Lotion: | | |
| Compound of Example 8 | 0.100 | g |
| Polyethylene glycol (PEG 400) | 69.900 | g |
| 95% ethanol | 30.000 | g |
| (f) Nonionic oil-in-water cream: | | |
| Compound of Example 5 | 1.000 | g |
| Cetyl alcohol | 4.000 | g |
| Glyceryl monostearate | 2.500 | g |
| PEG-5O stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water | qs 100 | g |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for regulating the size of sebaceous glands, comprising administering to a subject in need of such treatment, a thus effective amount of at least one activator of PPAR type receptors.

2. The regime or regimen as defined by claim 1, said effective amount of at least one activator of PPAR type receptors inhibiting the production of sebum by the sebaceous glands.

3. The regime or regimen as defined by claim 1, said at least one activator of PPAR type receptors having, for at least one of the subtypes PPAR α, γ or δ, a dissociation constant Kdapp of less than or equal to 500 nM.

4. The regime or regimen as defined by claim 3, said dissociation constant Kdapp being less than or equal to 100 nM.

5. The regime or regimen as defined by claim 1, said at least one activator of PPAR type receptors having, for at least the subtype PPAR-γ, a dissociation constant Kdapp of less than or equal to 500 nM.

6. The regime or regimen as defined by claim 5, said dissociation constant Kdapp being less than or equal to 100 nM.

7. The regime or regimen as defined by claim 5, said at least one activator of PPAR-γ type receptors being specific.

8. The regime or regimen as defined by claim 1, comprising topically applying a composition containing said effective amount of at least one activator of PPAR type receptors onto the skin, mucous membranes or keratin fibers of said subject in need of such treatment.

9. A regime or regimen for treating perioral dermatitis, a pathology associated with hyperplasia of the sebaceous glands, hereditary hyperplasia of the sebaceous glands, the overproduction of sebum associated with a hormonal disorder, or hyperandrogeny of endocrine origin, comprising administering to a subject in need of such treatment, a thus effective amount of at least one activator of PPAR type receptors.

10. A regime or regimen for treating greasy skin or a skin prone to dandruff, comprising administering to a subject in need of such treatment, a thus effective amount of at least one activator of PPAR type receptors.

11. The regime or regimen as defined by claim 10, comprising topically applying said at least one activator of PPAR type receptors onto the skin, mucous membranes or keratin fibers of said subject in need of such treatment.

12. The regime or regimen as defined by claim 1, said at least one activator of PPAR type receptors comprising:
1. 5-{4-2-methylpyrid-2-ylamino)ethoxy]benzyl}-thiazolidine-2,4-dione,
2. N-methyl-N-[4′-(2,4-dioxothiazolidin-5-ylmethyl)-biphenyl-3-ylmethyl)-6-(2-methoxyethoxymethoxy)-naphthalene-2-carboxamide,
3. (S)-3-[3′-(3-heptyl-1-methylureido)biphenyl-4-yl]-2-[2-(1-phenylmethanoyl)phenylamino]propionic acid,
4. N-methyl-N-[4′-(2,4-dioxothiazolidin-5-ylmethyl)-biphenyl-3-ylmethyl]-6-propoxynaphthalene-2-carboxamide,
5. (S)-2-ethoxy-3-{3′-[(methyloctanoylamino)methyl]biphenyl-4-yl}propionic acid,
6. 2-{3′-[({1-[6-(2-methoxyethoxymethoxy)naphthalene-2-yl]methanoyl}methylamino)methyl]biphenyl-4-ylamino}-benzoic acid,
7. (S)-3-{3′-[3-(4-dimethylaminophenyl)-1-methyl-ureido]biphenyl -4-yl}-2-[2-(1-phenylmethanoyl)phenyl-amino]propionic acid,
8. 2-(4-{2-[3-(2,4-difluorophenyl)-1-heptylureido]ethyl}phenylsulfanyl)-2-methylpropionic acid,
9. 1-[4′-(2,4-dioxothiazolidin-5-ylmethyl)biphenyl-3-yl]-3-heptyl-1-methylurea,
10. {3-[4-(3-cyclohexyl-1-phenethylureido)phenyl sulfanyl]phenyl}acetic acid,
11. {3-[4-(3-hexyl-1-phenethylureido)phenylsulfanyl]-phenyl}acetic acid,
12. {3-[4-(1-butyl-3-cyclohexylureido)phenylsulfanyl]-phenyl)acetic acid,
13. {3-[4-(3-benzyl-1-butylureido)phenylsulfanyl]-phenyl}acetic acid,
14. (S)-3-[3′-(3-heptyl-1-methylureido)biphenyl-4-yl]-2-[2-(1-phenylmethanoyl)phenylamino]propionic acid, and/or
15. ethyl (S)-3-[3′-(3-heptyl-1-methylureido)biphenyl-4-yl]-2-[2-(1-phenylmethanoyl)phenylamino]propionate.

13. A cosmetic/dermatological composition suited for regulating the size of sebaceous glands, comprising a thus effective amount of at least one activator of PPAR type receptors, formulated into a physiologically acceptable medium therefor.

14. The cosmetic/dermatological composition as defined by claim 13, comprising from 0.001% to 10% by weight of said at least one activator of PPAR type receptors.

15. The cosmetic/dermatological composition as defined by claim 13, comprising from 0.01% to 1% by weight of said at least one activator of PPAR type receptors.

16. The cosmetic/dermatological composition as defined by claim 13, comprising a matting agent.

17. The cosmetic/dermatological composition as defined by claim 13, comprising an anti-dandruff agent.

18. The cosmetic/dermatological composition as defined by claim 13, formulated for topical application onto the skin, mucous membranes or keratin fibers.

19. The cosmetic/dermatological composition as defined by claim 13, said at least one activator of PPAR type receptors comprising:
1. 5-{4-2-methylpyrid-2-ylamino)ethoxy]benzyl}-thiazolidine-2,4-dione,
2. N-methyl-N-[4′-(2,4-dioxothiazolidin-5-ylmethyl)-biphenyl-3-ylmethyl)-6-(2-methoxyethoxymethoxy)-naphthalene-2-carboxamide,
3. (S)-3-[3′-(3-heptyl-1-methylureido)biphenyl-4-yl]-2-[2-(1-phenylmethanoyl)phenylamino]propionic acid,
4. N-methyl-N-[4′-(2,4-dioxothiazolidin-5-ylmethyl)-biphenyl-3-ylmethyl]-6-propoxynaphthalene-2-carboxamide,
5. (S)-2-ethoxy-3-{3′-[(methyloctanoylamino)methyl]biphenyl-4-yl}propionic acid,
6. 2-{3′-[({1-[6-(2-methoxyethoxymethoxy)naphthalene-2-yl]methanoyl}methylamino)methyl]biphenyl-4-ylamino}-benzoic acid,
7. (S)-3-{3′-[3-(4-dimethylaminophenyl)-1-methyl-ureido]biphenyl -4-yl}-2-[2-(1-phenylmethanoyl)phenyl-amino]propionic acid,
8. 2-(4-{2-[3-(2,4-difluorophenyl)-1-heptylureido]ethyl}phenylsulfanyl)-2-methylpropionic acid,
9. 1-[4′-(2,4-dioxothiazolidin-5-ylmethyl)biphenyl-3-yl]-3-heptyl-1-methylurea,
10. {3-[4-(3-cyclohexyl-1-phenethylureido)phenyl sulfanyl]phenyl}acetic acid,
11. {3-[4-(3-hexyl-1-phenethylureido)phenylsulfanyl]-phenyl}acetic acid,
12. {3-[4-(1-butyl-3-cyclohexylureido)phenylsulfanyl]-phenyl)acetic acid,
13. {3-[4-(3-benzyl-1-butylureido)phenylsulfanyl]-phenyl}acetic acid,
14. (S)-3-[3′-(3-heptyl-1-methylureido)biphenyl-4-yl]-2-[2-(1-phenylmethanoyl)phenylamino]propionic acid, and/or
15. ethyl (S)-3-[3′-(3-heptyl-1-methylureido)biphenyl-4-yl]-2-[2-(1-phenylmethanoyl)phenylamino]propionate.

20. The cosmetic/dermatological composition as defined by claim 13, formulated for oral or parenteral administration.
